(19) United States
(12) Patent Application Publication
(10) Pub. No.: US 2019/0192795 A1
(43) Pub. Date: Jun. 27, 2019

(54) EXPIRATORY FLOW LIMITATION DETECTION VIA FLOW RESISTOR ADJUSTMENT

(71) Applicant: KONINKLIJKE PHILIPS N.V., EINDHOVEN (NL)

(72) Inventors: Francesco VICARIO, BOSTON, MA (US); William Anthony TRUSCHEL, OAKMONT, PA (US)

(21) Appl. No.: 16/224,897

(22) Filed: Dec. 19, 2018

Related U.S. Application Data
(60) Provisional application No. 62/610,736, filed on Dec. 27, 2017.

(51) Int. Cl. A61M 16/00; A61B 5/087

(52) U.S. Cl. CPC A61M 16/0003; A61B 5/4836; A61B 5/0871; A61M 16/024

(57) ABSTRACT

A system for detecting EFL of a patient is provided. The system comprises an inhalation passage; an exhalation passage; a sensor for measuring flow-volume information of the exhaled air through an exhalation passage; a flow resistor positioned in the exhalation passage and being adjustable to provide an exhalation resistance in the exhalation passage; and a computer system. In one embodiment, one or more physical processors of the computer system are programmed with computer program instructions which, when executed cause the computer system to: determine a reference expiratory flow-volume curve using flow-volume information of the exhaled air through the exhalation passage when a reference exhalation resistance is provided by the flow resistor in the exhalation passage; and determine a perturbed expiratory flow-volume curve using flow-volume information of the exhaled air through the exhalation passage when a lowered exhalation resistance is provided by the flow resistor in the exhalation passage.

### CROSS-REFERENCE TO RELATED APPLICATIONS

This patent application claims the priority benefit under 35 U.S.C. § 119(e) of U.S. Provisional Application No. 62/610,736, filed on Dec. 27, 2017, the contents of which are herein incorporated by reference.

### BACKGROUND

### 1. Field of the Invention

The present disclosure pertains to a method and a system for detecting an Expiratory Flow Limitation (EFL) of a patient.

### 2. Description of the Related Art

Chronic Obstructive Pulmonary Disease (COPD) is a public health problem. It is the fourth leading cause of chronic morbidity and mortality in the United States, affecting over 24 million Americans. It is the third leading cause of death in the United States after heart disease and cancer and, by 2020, it is projected to be the third leading cause of death worldwide. The increased mortality is driven by the expanding epidemic of smoking and the aging population worldwide.

COPD is an umbrella term used to describe progressive lung diseases including emphysema, chronic bronchitis, non-reversible asthma, and some forms of bronchiectasis. COPD is characterized by increasing breathlessness. Patients with COPD may have difficulty exhaling because of the deterioration of their lung tissue or the inflammation of their airway walls. This condition is commonly referred to as Expiratory Flow Limitation (EFL) and it affects the quality of life and can ultimately contribute to acute respiratory failure. As an example, the expiratory flow limitation relates to a physiological condition where a person's airways partially collapse due to a loss of their elastic recoil due to parenchymal destruction or to some other form of airway obstruction. “The definition of EFL implies that a further increase in transpulmonary pressure will cause no further increase in expiratory flow,” as disclosed in N. G. Koulouris et al., “Methods for assessing expiratory flow limitation during tidal breathing in COPD patients,” Pulmonary Medicine, vol. 2012, doi:10.1155/2012/234145. The expiratory flow limitation of a subject is determined by detecting via one or more sensors when flow ceases to increase despite increasing expiratory effort. A patient with EFL cannot increase his flow rate by force and often increases his dynamic volume towards Total Lung Capacity, TLC (dynamic hyperinflation) causing muscle fatigue. Also, a patient with EFL has a lower exercise tolerance and chronic dyspnea, leading to an unhealthy, sedentary life-style.

Common treatments for EFL are the application of positive airway pressure and/or pharmaceuticals.

With patients in the ICU, EFL is detected with a manual maneuver. A clinician/respiratory therapist exerts force on the patient's abdomen at the onset of exhalation. That is, the physician may simply press on the ventilated patient during expiration and determine if there is or there is not an increase in flow. This force causes an increase in the pressure difference between the lungs and mouth that should drive the exhalation flow. If the patient has EFL, the exhalation flow does not increase. This manual chest compression technique i) does not require patient collaboration, ii) lacks repeatability (manual operation), and iii) needs skilled personnel. The manual chest compression technique is also not applicable for a chronic patient at home.

Other techniques for the detection of EFL are the ΔXrs with forced oscillation technique (FOT) and the negative expiratory pressure (NEP) method.

For example, the NEP method i) does not require patient collaboration, ii) requires negative pressure (or at least positive pressure), and iii) can result in upper airway artifacts. The FOT method i) does not require patient collaboration and ii) requires generation of pressure oscillations.

Further, the FOT and NEP method are available as stand-alone devices or as part of multifunctional spirometers. The FOT and NEP method are typically used for non-ventilated patients. The NEP method is conceptually similar to the application of pressure on the abdomen. It replaces the increasing pressure in the lungs from abdominal compression with negative pressure applied at the mouth. The ΔXrs with the FOT method relies on the change in the reactance of the respiratory system when EFL occurs. In order to “measure” the reactance, a forced sinusoidal pressure signal is applied. In primary care settings, the ratio between FEV1 (forced expiratory volume in 1 second) and FVC (functional vital capacity) obtained from spirometry is typically used.

Therefore, an improved system and method for detecting an Expiratory Flow Limitation (EFL) of a patient is needed.

### SUMMARY OF THE INVENTION

Accordingly, it is an object of one or more embodiments of the present patent application to provide a system for detecting an Expiratory Flow Limitation (EFL) of a patient. The system comprises an inhalation passage that brings inhaled air to the patient; an exhalation passage that takes exhaled air away from the patient; a sensor for measuring flow-volume information of the exhaled air through the exhalation passage; a flow resistor positioned in the exhalation passage and being adjustable to provide an exhalation resistance in the exhalation passage; and a computer system that comprises one or more physical processors operatively connected with the sensor and the flow resistor. In one embodiment, the one or more physical processors are programmed with computer program instructions which, when executed cause the computer system to: determine a reference expiratory flow-volume curve using flow-volume information of the exhaled air through the exhalation passage when a reference exhalation resistance is provided by the flow resistor in the exhalation passage; adjust the flow resistor to lower the exhalation resistance below the reference exhalation resistance; determine a perturbed expiratory flow-volume curve using flow-volume information of the exhaled air through the exhalation passage when the lowered exhalation resistance is provided by the flow resistor in the exhalation passage; and detect the EFL of the patient based on (i) the determined perturbed expiratory flow-volume curve and (ii) the determined reference expiratory flow-volume curve.

It is yet another aspect of one or more embodiments of the present patent application to provide a method for detecting an Expiratory Flow Limitation (EFL) of a patient. The method is implemented by a computer system that comprises one or more physical processors executing computer program instructions which, when executed, perform the method. The method comprises obtaining, from one or more sensors, flow-volume information of exhaled air through an exhalation passage; determining, by the computer system, a reference expiratory flow-volume curve using flow-volume information of the exhaled air through the exhalation passage when a reference exhalation resistance is provided by a flow resistor in the exhalation passage; adjusting the flow resistor to lower the exhalation resistance below the reference exhalation resistance; determining, by the computer system, a perturbed expiratory flow-volume curve using flow-volume information of the exhaled air through the exhalation passage when the lowered exhalation resistance is provided by the flow resistor in the exhalation passage; and detecting, by the computer system, the Expiratory Flow Limitation (EFL) of the patient based on (i) the determined perturbed expiratory flow-volume curve (ii) the determined reference expiratory flow-volume curve.

It is yet another aspect of one or more embodiments to provide a system for detecting an Expiratory Flow Limitation (EFL) of a patient. The system comprises a means for executing machine-readable instructions with at least one physical processor. The machine-readable instructions comprises obtaining, from one or more sensors, flow-volume information of exhaled air through an exhalation passage; determining a reference expiratory flow-volume curve using flow-volume information of the exhaled air through the exhalation passage when a reference exhalation resistance is provided by a flow resistor in the exhalation passage; adjusting the flow resistor to lower the exhalation resistance below the reference exhalation resistance; determining a perturbed expiratory flow-volume curve using flow-volume information of the exhaled air through the exhalation passage when the lowered exhalation resistance is provided by the flow resistor in the exhalation passage; and detecting the Expiratory Flow Limitation (EFL) of the patient based on (i) the determined perturbed expiratory flow-volume curve (ii) the determined reference expiratory flow-volume curve.

These and other objects, features, and characteristics of the present patent application, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present patent application.

### BRIEF DESCRIPTION OF THE DRAWINGS

FIG. 1 is an exemplary system for detecting an Expiratory Flow Limitation (EFL) of a patient in accordance with an embodiment of the present patent application;

FIG. 2 is an exemplary system for detecting EFL of the patient in accordance with another embodiment of the present patent application;

FIG. 3 is an exemplary system for detecting EFL of the patient in accordance with another embodiment of the present patent application;

FIG. 4 is an exemplary system for detecting EFL of the patient in accordance with another embodiment of the present patent application;

FIG. 5 shows a graphical illustration of an exemplary exhalation resistance reduction on select breaths in the system for detecting the EFL of the patient in accordance with an embodiment of the present patent application;

FIG. 6 shows exemplary flow comparisons between perturbed breath flow-volume curves and reference breaths flow-volume curves obtained from the system for detecting the EFL of the patient in accordance with an embodiment of the present patent application;

FIG. 7 shows exemplary EFL detection by reduction of exhalation resistance in the system for detecting the EFL of the patient and corresponding treatment to abolish EFL using the same system in accordance with an embodiment of the present patent application; and

FIG. 8 shows an exemplary method for detecting EFL of the patient and corresponding abolishment of the EFL in accordance with an embodiment of the present patent application.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other. As used herein, the term “or” means “and/or” unless the context clearly dictates otherwise.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The present patent application provides a system 100 for detecting an Expiratory Flow Limitation (EFL) of a patient. System 100 comprises an inhalation passage 104 that brings inhaled air to the patient; an exhalation passage 108 that takes exhaled air away from the patient; a sensor 106 for measuring flow-volume information of the exhaled air through exhalation passage 108; a flow resistor 110 positioned in exhalation passage 108 and being adjustable to provide an exhalation resistance in exhalation passage 108; and a computer system 102 that comprises one or more physical processors operatively connected with sensor 106 and flow resistor 110.

In one embodiment, the one or more physical processors of computer system 102 are programmed with computer program instructions which, when executed cause computer system 102 to: determine a reference expiratory flow-volume curve using flow-volume information of the exhaled air through exhalation passage 108 when a reference exhalation resistance is provided by flow resistor 110 in exhalation passage 108; adjust flow resistor 110 to lower the exhalation resistance below the reference exhalation resistance; determine a perturbed expiratory flow-volume curve using flow-volume information of the exhaled air through exhalation passage 108 when the lowered exhalation resistance is provided by flow resistor 110 in the exhalation passage; and detect the Expiratory Flow Limitation (EFL) of the patient based on i) the determined perturbed expiratory flow-volume curve and ii) the determined reference expiratory flow-volume curve.

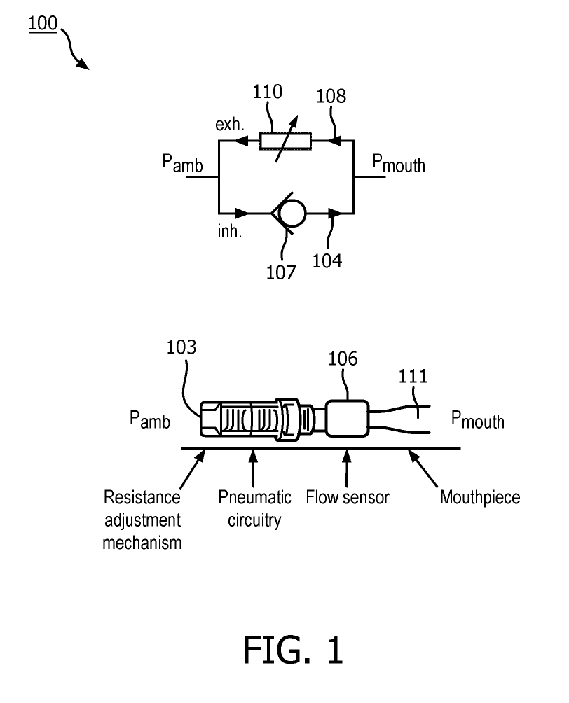

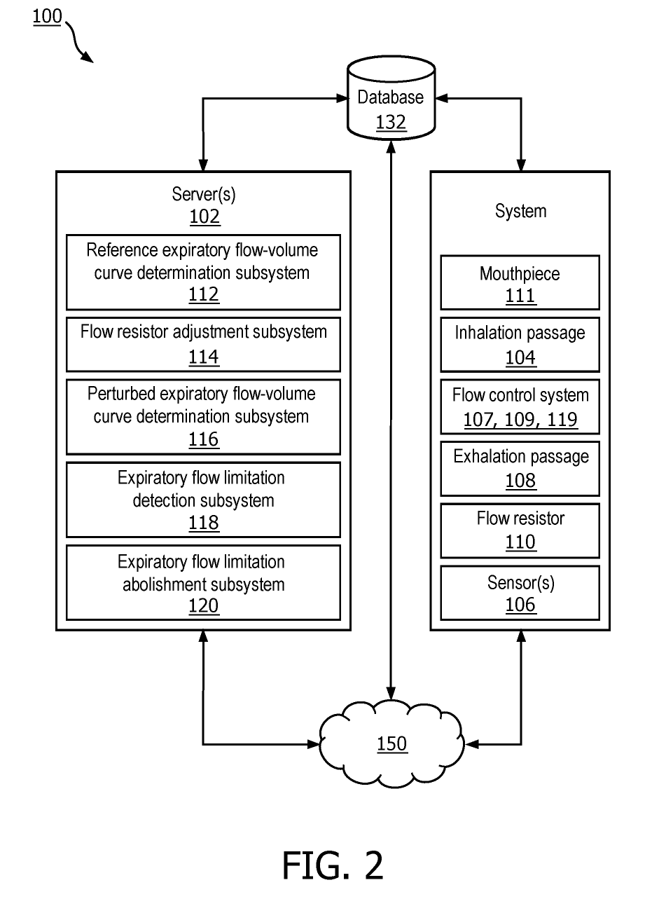

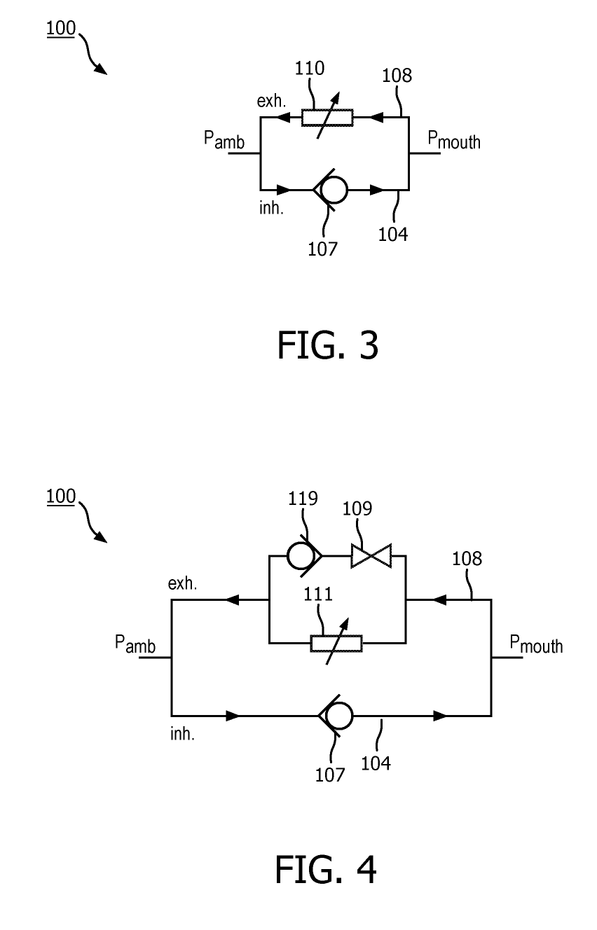

In one embodiment, system 100 is configured for facilitating expiratory flow limitation detection via automated adjustment of flow resistor 110 placed in exhalation passage 108. In one embodiment, a hand-held, low-cost, state-of-the-art lung function measurement instrument/system 100 that has been exclusively designed to detect the presence of EFL in COPD patients is disclosed in the present patent application. In one embodiment, system 100 includes a pneumatic circuit and device/system 100 as shown in FIG. 1. In one embodiment, system 100 includes a pneumatic circuit as shown in FIG. 3. In one embodiment, system 100 includes a pneumatic circuit as shown in FIG. 4. In one embodiment, system 100 may be a system as shown in FIG. 2. In one embodiment, system 100 includes an adjustable Positive Expiratory Pressure (PEP) therapy or PEP spirometer system/device.

In one embodiment, referring to FIGS. 1-4, system 100 includes tubing or conduit portions that form inhalation passage 104. In one embodiment, inhalation passage 104 is configured to bring inhaled air to the patient.

In one embodiment, system 100 includes an inhalation valve 107 that is configured to be in communication with inhalation passage 104. In one embodiment, inhalation valve 107 includes a check valve. In one embodiment, inhalation valve 107 includes a ball valve. In one embodiment, inhalation valve 107 includes a one-way valve. In one embodiment, inhalation valve 107 includes a controllable valve. In one embodiment, inhalation valve 107 may be any valve assembly that is configured to bring inhaled air to the patient (i.e., to allow a patient to breath in). In one embodiment, the direction of flow of gas/air in inhalation passage 104 is opposite to that in exhalation passage 108 as shown by the arrows in FIGS. 1, 3, and 4.

In one embodiment, each respiration cycle generally includes an inhalation phase and an exhalation phase. In one embodiment, during the inhalation phase, inhalation valve 107 is open and exhalation valve 109 is closed. That is, during the inhalation phase, a flow of gas (e.g., at ambient pressure P_{amb}) passes through the open inhalation valve 107, through inhalation passage 104 into the patient's mouth (airways and lungs). In one embodiment, air at ambient pressure P_{amb} is drawn into the inhalation passage 104, for example, through inhalation valve 107. That is, inhalation valve 107 is configured to open to allow the patient to inhale substantially resistance free. In one embodiment, the flow of gas is drawn through an inlet opening 103 of system 100. During the exhalation phase, air is prevented from exiting through the inlet opening 103 by the closing of the inhalation valve 107.

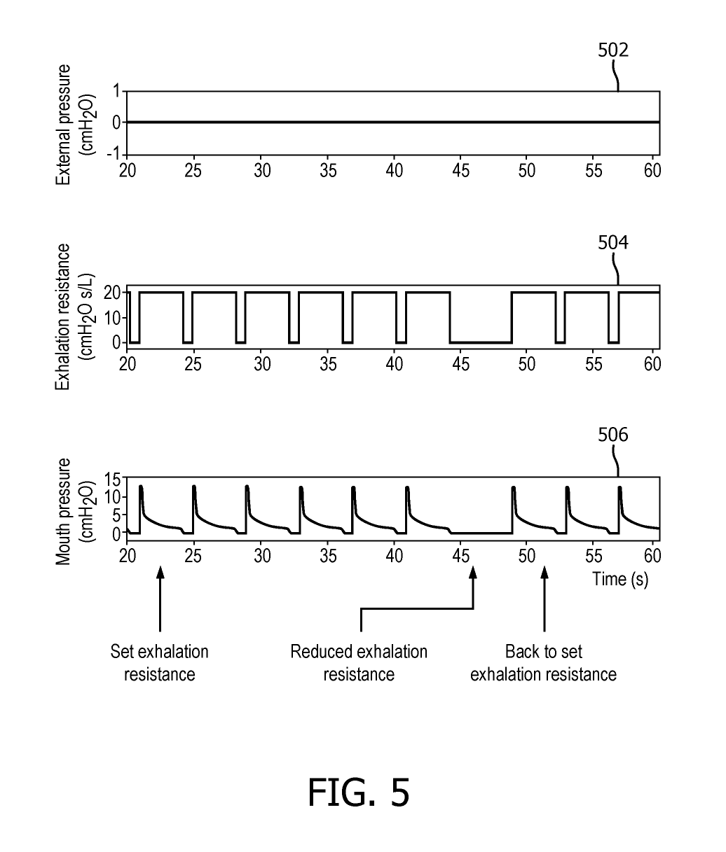

In one embodiment, during the inhalation phase, the pressure at the mouth of the patient is approximately equal to the ambient pressure, P_{amb} (i.e., 0 cm H₂0), whereas, during the exhalation phase, air goes through resistive element or resistor 110 that causes a drop in pressure, i.e., the pressure at the mouth Pₘₒᵤₜₕ will be higher than the ambient pressure, P_{amb} (as illustrated in FIG. 5).

In one embodiment, system 100 is also configured to detect the start and end of the exhalation phase. In one embodiment, system 100 includes an algorithm to detect the start and end of the exhalation phase.

In one embodiment, system 100 includes tubing or conduit portions that form exhalation passage 108. In one embodiment, exhalation passage 108 is configured to take the exhaled air away from the patient. In one embodiment, as shown in FIG. 4, exhalation passage 108 of system 100 includes a check valve 119. In one embodiment, as shown in FIG. 4, exhalation passage 108 includes an on-off valve 109. In one embodiment, as shown in FIG. 4, exhalation passage 108 includes both check valve 119 and on-off valve 109. In one embodiment, exhalation valve 109/119 are configured to be in communication with exhalation passage 108. In one embodiment, exhalation valve 109/119 may be any valve assembly that is configured to control/allow the flow of the exhaled air from the patient to escape to atmosphere through exhalation passage 108. In one embodiment, exhalation valve 109 includes a solenoid or electromechanical operated valve. In one embodiment, exhalation valve 119 includes a ball valve. In one embodiment, exhalation valve 119 includes a check valve. In one embodiment, exhalation valve 119 includes a one-way valve.

In one embodiment, system 100 is configured to control a variable resistance during the patient's exhalation. In one embodiment, system 100 is also configured to remove a variable resistance during the patient's exhalation as will be described in detail below.

In one embodiment, system 100 includes flow resistor 110 that is positioned in exhalation passage 108. In one embodiment, flow resistor 110 may include a flow resistive element. In one embodiment, flow resistor 110 may be an electro-flow resistive element. In one embodiment, flow resistor 110 may be a mechanical flow resistive element. In one embodiment, flow resistor 110 may be an electro-mechanical flow resistive element. In one embodiment, flow resistor 110 may be any flow resistive element that is configured to provide an exhalation resistance in exhalation passage 108.

In one embodiment, flow resistor 110 is configured to be adjustable to provide an exhalation resistance in exhalation passage 108. In one embodiment, the exhalation resistance provided by flow resistor 110 is configured to be decreased on select breaths in order to increase the exhalation pressure drive and mimic the abdomen compression maneuver as will be described in detail below.

In one embodiment, flow resistor 110 is configured to be manually actuated and/or adjustable. In one embodiment, flow resistor 110 is configured to be mechanically actuated and/or adjustable.

In one embodiment, flow resistor 110 is configured to be operatively connected with one or more physical processors of computer system 102. In one embodiment, as will be described in detail below, flow resistor 110 is configured to be adjustable by computer system 102. In one embodiment, as will be described in detail below with respect to system 100 in FIG. 2, flow resistor 110 is configured to be adjustable by a flow resistor subsystem 114 of computer system 102.

In one embodiment, system 100 includes sensor 106 to measure the flow of the gas/air exhaled by the patient. In one embodiment, sensor 106 is configured to measure and provide respiratory parameters such as flow rate, flow-volume, etc. In one embodiment, sensor 106 is configured to measure flow-volume information of the exhaled air through exhalation passage 108. In one embodiment, sensor 106 is configured to measure volumetric flow rate of the exhaled air through exhalation passage 108.

In one embodiment, sensor 106 is a flow sensor. In one embodiment, sensor 106 is a pressure sensor. In one embodiment, sensor 106 includes a flow sensor and a pressure sensor.

In one embodiment, sensor 106 is in fluid communication with exhalation passage 108. During the exhalation/expiration phase, sensor 106 is configured to measure the flow through and/or pressure in exhalation passage 108. In one embodiment, sensor 106 may be calibrated to sense the beginning of exhalation/expiration phase and to begin the sensing procedure. In one embodiment, sensor 106 is configured to be operatively connected with one or more physical processors of computer system 102. In one embodiment, sensor 106 is configured to be operatively connected with reference expiratory flow-volume curve determination subsystem 112 and perturbed expiratory flow-volume curve determination subsystem 116 of computer system 102. In one embodiment, sensor 106 is configured to be operatively connected with a database (e.g., database 132) to save the flow-volume information of the exhaled air through exhalation passage 108 into the database. Saved flow-volume information of the exhaled air through exhalation passage 108 may later retrieved from the database as needed.

In one embodiment, system 100 includes a mouthpiece 111 through which the patient breathes into system 100. In one embodiment, mouthpiece 111 may be of the type where the patient takes the part of mouthpiece 111 into his/her mouth. In one embodiment, system 100 includes a mask 111 through which the patent breathes into system 100. In one embodiment, mask or mouthpiece 111 is configured to be removably connected to system 100. In one embodiment, the patient discharges expiratory air into mask or mouthpiece 111.

FIG. 2 shows system 100 for detecting EFL of a patient, in accordance with one or more embodiments. As shown in FIG. 2, system 100 may comprise server 102 (or multiple servers 102). Server 102 may comprise reference expiratory flow-volume curve determination subsystem 112, flow resistor adjustment subsystem 114, perturbed expiratory flow-volume curve determination subsystem 116, Expiratory Flow Limitation (EFL) detection subsystem 118, Expiratory Flow Limitation abolishment subsystem 120, or other components or subsystems.

In one embodiment, Expiratory Flow Limitation abolishment subsystem 120 is optional. It should be appreciated that the description of the functionality provided by the different subsystems 112-120 described herein is for illustrative purposes, and is not intended to be limiting, as any of subsystems 112-120 may provide more or less functionality than is described. For example, one or more of subsystems 112-120 may be eliminated, and some or all of its functionality may be provided by other ones of subsystems 112-120. As another example, additional subsystems may be programmed to perform some or all of the functionality attributed herein to one of subsystems 112-120.

In one embodiment, reference expiratory flow-volume curve determination subsystem 112 is configured to determine a reference expiratory flow-volume curve using flow-volume information of the exhaled air through exhalation passage 108 when a reference exhalation resistance is provided by flow resistor 110 in exhalation passage 108.

In one embodiment, the set or reference exhalation resistance may be obtained by clinical testing. In one embodiment, the set or reference exhalation resistance may be obtained using data analytics. In one embodiment, the set or reference exhalation resistance may be obtained from research publications. In one embodiment, the reference or set expiratory resistance may be saved into a database (e.g., database 132) and retrieved from the database as needed. In one embodiment, subsystem of system 100 may continuously update/modify the reference or set expiratory resistance. In one embodiment, the set or reference exhalation resistance is configured such that the resulting positive expiratory pressure is constant or approximately so.

In one embodiment, reference expiratory flow-volume curve determination subsystem 112 may obtain information associated with exhalation passage 108 when a reference exhalation resistance is provided by flow resistor 110 in exhalation passage 108. In one embodiment, the information may include flow-volume information, flow information, pressure information, or any other related information. In one embodiment, the flow-volume information of the patient may include information about flow-volume in exhalation passage 108 when a reference exhalation resistance is provided by flow resistor 110 in exhalation passage 108. In one embodiment, the flow information of the patient may include information about flow through exhalation passage 108 when a reference exhalation resistance is provided by flow resistor 110 in exhalation passage 108. In one embodiment, the pressure information of the patient may include information about pressure in exhalation passage 108 when a reference exhalation resistance is provided by flow resistor 110 in exhalation passage 108.

As another example, the information may be obtained from one or more monitoring devices (e.g., flow monitoring device, pressure monitoring device, or other monitoring devices). In one embodiment, one or more monitoring devices and associated sensors 106 may be configured to monitor flow-volume in exhalation passage 108 when a reference exhalation resistance is provided by flow resistor 110 in exhalation passage 108. In one embodiment, one or more monitoring devices and associated sensors 106 may be configured to monitor flow through exhalation passage 108 when a reference exhalation resistance is provided by flow resistor 110 in exhalation passage 108. In one embodiment, one or more monitoring devices and associated sensors 106 may be configured to monitor pressure in exhalation passage 108 when a reference exhalation resistance is provided by flow resistor 110 in exhalation passage 108. These monitoring devices may include one or more sensors 106, such as pressure sensors, pressure transducers, flow rate sensors, flow sensors, volume sensors, or other sensors. Sensors may, for instance, be configured to obtain information of the patient (e.g., pressure, flow, flow-volume, volume, or any other related parameters) or other information related to exhalation passage 108 when a reference exhalation resistance is provided by flow resistor 110 in exhalation passage 108.

In one scenario, a monitoring device may obtain information (e.g., based on information from one or more sensors 106), and provide information to a computer system (e.g., comprising server 102) over a network (e.g., network 150) for processing. In another scenario, upon obtaining the information, the monitoring device may process the obtained information, and provide processed information to the computer system over a network (e.g., network 150). In yet another scenario, the monitoring device may automatically provide information (e.g., obtained or processed) to the computer system (e.g., comprising server 102).

In one embodiment, reference expiratory flow-volume curve determination subsystem 112 is configured to determine the reference expiratory flow-volume curve from the obtained flow-volume information when a reference exhalation resistance is provided by flow resistor 110 in exhalation passage 108. That is, reference expiratory flow-volume curve determination subsystem 112 is configured to analyze information/data from a device's flow and pressure sensors and calculate or determine reference expiratory flow-volume curve based on the sensor data/information. In one embodiment, reference expiratory flow-volume curve determination subsystem 112 may be configured to determine the reference expiratory flow-volume curve directly from the flow and pressure signals.

In one embodiment, flow resistor adjustment subsystem 114 is configured to be operatively associated with flow resistor 110. In one embodiment, flow resistor adjustment subsystem 114 is configured to control a variable resistance during the patient's exhalation. In one embodiment, flow resistor adjustment subsystem 114 is also configured to remove a variable resistance during the patient's exhalation.

In one embodiment, flow resistor adjustment subsystem 114 is configured to adjust flow resistor 110 to the reference exhalation resistance. In one embodiment, flow resistor adjustment subsystem 114 is configured to adjust flow resistor 110 to lower the exhalation resistance below the reference exhalation resistance. In one embodiment, flow resistor adjustment subsystem 114 is configured to decrease (or reduce/drop) the exhalation resistance on select breaths. In one embodiment, flow resistor adjustment subsystem 114 is configured to adjust/change the exhalation resistance, for example, to abolish EFL. In one embodiment, flow resistor adjustment subsystem 114 is configured to increase the reference exhalation resistance on a select breath.

In one embodiment, the configuration, operation and structure of perturbed expiratory flow-volume curve determination subsystem 116 are similar that of reference expiratory flow-volume curve determination subsystem 112 except for the differences noted below. In one embodiment, perturbed expiratory flow-volume curve determination subsystem 116 is configured to determine a perturbed expiratory flow-volume curve using flow-volume information of the exhaled air through exhalation passage 108 when the lowered exhalation resistance (i.e., below the set/reference exhalation resistance) is provided by flow resistor 110 in exhalation passage 108.

In one embodiment, perturbed expiratory flow-volume curve determination subsystem 116 may obtain information associated with exhalation passage 108 when the lowered exhalation resistance (i.e., below the set/reference exhalation resistance) is provided by flow resistor 110 in exhalation passage 108. In one embodiment, the information may include flow-volume information, flow information, pressure information, or any other related information when a lowered exhalation resistance (i.e., below the set/reference exhalation resistance) is provided by flow resistor 110 in exhalation passage 108. In one embodiment, one or more monitoring devices and associated sensors 106 may be configured to monitor flow-volume, flow, pressure, or other related information in exhalation passage 108 when a lowered exhalation resistance (i.e., below the set/reference exhalation resistance) is provided by flow resistor 110 in exhalation passage 108.

In one embodiment, perturbed expiratory flow-volume curve determination subsystem 116 is configured to determine the perturbed expiratory flow-volume curve from the obtained flow-volume information when a lowered exhalation resistance (i.e., below the set/reference exhalation resistance) is provided by flow resistor 110 in exhalation passage 108. That is, perturbed expiratory flow-volume curve determination subsystem 116 is configured to analyze information/data from a device's flow and pressure sensors and calculate or determine perturbed expiratory flow-volume curve based on the sensor data/information. In one embodiment, perturbed expiratory flow-volume curve determination subsystem 116 may be configured to determine the perturbed expiratory flow-volume curve directly from the flow and pressure signals.

In one embodiment, system 100 includes an algorithm to compute exhalation flow-volume curves. In one embodiment, reference expiratory flow-volume curve determination subsystem 112 and perturbed expiratory flow-volume curve determination subsystem 116 of system 100 each includes an algorithm to compute the exhalation flow-volume curves. That is, in one embodiment, reference expiratory flow-volume curve determination subsystem 112 is configured to determine, using an algorithm, a reference expiratory flow-volume curve using flow-volume information of the exhaled air through exhalation passage 108 when a reference exhalation resistance is provided by flow resistor 110 in exhalation passage 108. In one embodiment, perturbed expiratory flow-volume curve determination subsystem 116 is configured to determine, using an algorithm, a perturbed expiratory flow-volume curve using flow-volume information of the exhaled air through exhalation passage 108 when the lowered exhalation resistance (i.e., below the set/reference exhalation resistance) is provided by flow resistor 110 in exhalation passage 108.

In one embodiment, Expiratory Flow Limitation (EFL) detection subsystem 118 is configured to detect the Expiratory Flow Limitation (EFL) of the patient based on i) the determined perturbed expiratory flow-volume curve (e.g., from reference expiratory flow-volume curve determination subsystem 112) and ii) the determined reference expiratory flow-volume curve (e.g., from perturbed expiratory flow-volume curve determination subsystem 116). In one embodiment, Expiratory Flow Limitation (EFL) detection subsystem 118 is configured to detect the Expiratory Flow Limitation (EFL) of the patient by comparing the determined perturbed expiratory flow-volume curve (e.g., from reference expiratory flow-volume curve determination subsystem 112) with the determined reference expiratory flow-volume curve (e.g., from perturbed expiratory flow-volume curve determination subsystem 116).

In one embodiment, the presence of EFL is assessed by comparing the flow-volume curves of two breaths (i.e., a reference breath and a perturbed breath with exhalation resistance lower than the reference breath).

In one embodiment, the reference expiratory flow-volume curve and the perturbed expiratory flow-volume curve are displayed to the caregiver for a visual assessment of the breath. In one embodiment, the classification (i.e., EFL vs. no EFL) is done by the caregiver.

In one embodiment, system 100 also includes a user interface and/or other components. In one embodiment, the user interface is configured to provide an interface between system 100 and the patient/caregiver/physician. In one embodiment, the reference expiratory flow-volume curve and the perturbed expiratory flow-volume curve are displayed to the caregiver/physician via the user interface. In one embodiment, the caregiver/physician may specify one or more PEP therapy regimes that are to be delivered to the patient using the user interface. Examples of interface devices suitable for inclusion in the user interface comprise a keypad, buttons, switches, a keyboard, knobs, levers, a display screen, a touch screen, speakers, a microphone, an indicator light, an audible alarm, a printer, a tactile feedback device, and/or other interface devices. In one embodiment, the user interface comprises a plurality of separate interfaces. In one embodiment, the user interface comprises at least one interface that is provided integrally with system 100.

In one embodiment, the classification (i.e., EFL vs. no EFL) is automated. In one embodiment, the classification (i.e., EFL vs. no EFL) is done by an algorithm run by one or more processors of computer system 102 within system 100. In one embodiment, the classification (i.e., EFL vs. no EFL) is done by an algorithm run by one or more processors of computer system 102 outside system 100. That is, system 100 includes an automatic algorithm to determine test result (i.e., EFL vs. no EFL). In one embodiment, the classification algorithm is configured to receive as an input the exhalation waveform of the breath with reduced exhalation resistance (i.e., perturbed breath) and of the breath preceding the perturbation (i.e., reference breath). That is, the classification algorithm is configured to receive as an input the reference expiratory flow-volume curve and the perturbed expiratory flow-volume curve.

In one embodiment, the one or more breaths preceding the perturbation breath are used to increase the robustness of the algorithm (e.g., by computing an average reference breath) and/or assessing whether the reference breath is sufficiently stable and repeatable so that its comparison with the perturbed breath is not affected by confounding factors. In one embodiment, the only factor that can change the flow waveform is the driving pressure.

In one embodiment, the classification algorithm is based on a single feature computed from the exhalation flow waveform or flow-volume curve. In one embodiment, the feature computed from the exhalation flow waveform or flow-volume curve includes the percentage of exhaled volume of air that occurs with the flow from the perturbed breath that is equal to the flow from the reference breath. In one embodiment, the exhalation flow-volume curve or volume waveform is computed by numerical integration of the measured exhalation flow-volume curve or flow waveform. In one embodiment, among the algorithm parameters to optimize are the threshold to determine whether the flows can be considered equal and the threshold in the percentage of exhaled volume to declare whether a breath is flow-limited or not. In one embodiment, the above-mentioned percentage is related to the automatic change in pressure required to abolish EFL.

In one embodiment, the classification algorithm based on multiple features computed from the exhalation flow-volume curve or exhalation flow waveform. In one embodiment, the features computed from the exhalation flow waveform or exhalation flow-volume curve include the percentage of exhaled volume with same flow (reference vs. perturbed breath), the exhaled volume (over the same time for the reference and perturbed breaths), the amplitude of the peak that typically occurs in the perturbed breath. In one embodiment, the classification algorithm is data-driven and as such it is trained on datasets that include both flow-limited and non-flow-limited breaths (i.e., machine learning), and then validated on an independent dataset (i.e., excluded from the training phase).

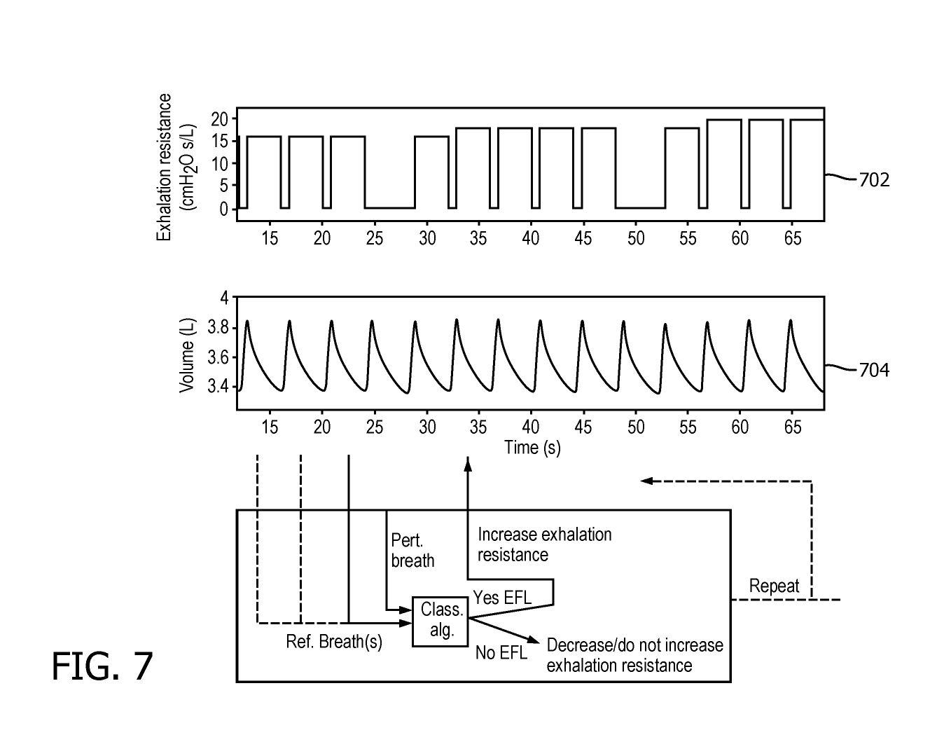

In one embodiment, as illustrated in FIG. 7, the input to the classification algorithm (e.g., expiratory flow limitation detection subsystem 118) includes the airflow waveforms/flow-volume curves from the perturbed breath and the airflow waveforms/flow-volume curves from the reference breaths (e.g., one or multiple breaths preceding the perturbed breath). In one embodiment, the airflow waveforms/flow-volume curves from the perturbed breath are sent to the classification algorithm (e.g., expiratory Flow Limitation Detection subsystem 118) from perturbed expiratory flow-volume curve determination subsystem 116. In one embodiment, the airflow waveforms/flow-volume curves from the reference breaths (e.g., one or multiple breaths preceding the perturbed breath) are sent to the classification algorithm (e.g., expiratory Flow Limitation Detection subsystem 118) from reference expiratory flow-volume curve determination subsystem 112.

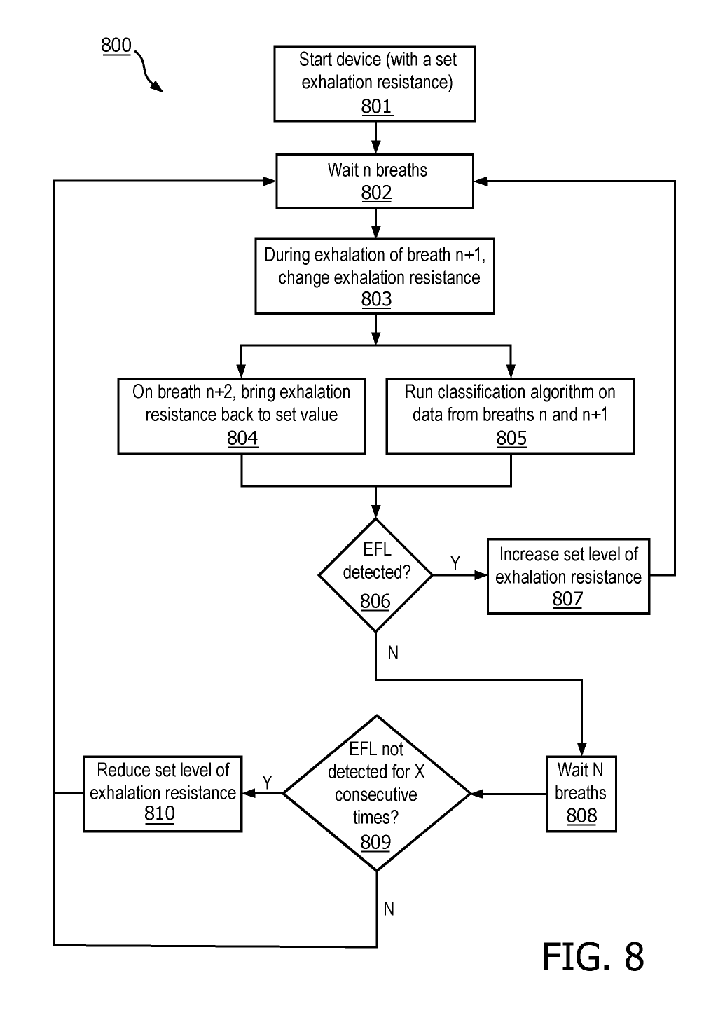

In one embodiment, Expiratory Flow Limitation (EFL) abolishment subsystem 120 is configured to abolish EFL upon its detection. In one embodiment, the exhalation resistance is also changed to abolish EFL. In one embodiment, system 100 may be used for real-time detection and abolishment of EFL. In one embodiment, as will be explained in detail with respect to FIG. 8, one or more processors of computer system 102 are configured to automatically increase or decrease the exhalation airway pressure upon detection of EFL in order to abolish EFL. In one embodiment, system 100 is configured to specifically detect EFL and possibly to treat it. Additionally, system 100 is configured for reduction of exhalation resistance on selected breaths.

In one embodiment, system 100 is configured to automatically adjust the PEP therapy to abolish EFL, upon detection of EFL. In one embodiment, the set exhalation resistance is increased and the procedures are repeated after some breaths. In one embodiment, once the EFL is abolished, the procedures are repeated to confirm the absence of EFL at regular time intervals or when changes in the breathing pattern are detected.

The schematics shown in FIG. 3 is only one possible embodiment. The implementation of the concept in FIG. 3 can follow different embodiments, for instance the one in FIG. 4, where an additional pathway or passage for exhalation is shown. This pathway/passage has two possible configurations: i) open (resistance-free), ii) closed (infinite resistance). In one embodiment, valve 109 is used to switch form one configuration to the other configuration. In one embodiment, normally (reference breaths), the additional pathway/passage is blocked (closed valve). In one embodiment, when the expiratory flow (EF) is needed, the additional pathway/passage is opened (open valve) to by-pass the exhalation resistance (perturbed breath).

FIG. 5 shows an example of the technique to detect EFL. FIG. 5 shows a graphical illustration of exemplary exhalation resistance reduction on select breaths in system 100. In one embodiment, on select breaths, the exhalation resistance is dropped. Such breaths are referred in this patent application as perturbed breaths.

In one embodiment, the exhalation flow-volume curve of a perturbed breath is compared with the exhalation flow-volume curve of one or more preceding breaths (reference breaths). FIG. 5 shows an example of exhalation resistance reduction on select breaths in system 100. In one embodiment, on the selected breaths, the exhalation resistance is by-passed in order to cause a lower mouth pressure Pₘₒᵤₜₕ during exhalation (i.e., higher expiratory drive).

FIG. 5 shows external pressure (e.g., measured in units of cm H₂0) on the left hand side Y-axis of graph 502 and time (e.g., measured in units of seconds) on X-axis of graph 502. For example, the external pressure is also referred to as the ambient pressure, P_{amb}. As can be seen from graph 502, the external pressure or ambient pressure, P_{amb} is maintained at a constant pressure of 0 cm H₂0 for the entire time period between 20 seconds and 60 seconds (as shown in the X-axis of the graph 502).

FIG. 5 shows exhalation resistance (e.g., measured in units of cm H₂0*s/L) on the left hand side Y-axis of graph 504 and time (e.g., measured in units of seconds) on X-axis of graph 504. For example, the exhalation resistance is the flow resistance applied by flow resistor 110 in exhalation path 108. Referring to graph 504, for the time period between 20 seconds and 45 seconds, the exhalation resistance is maintained at 20 cm H₂0*s/L. The exhalation resistance is then reduced/dropped from 20 cm H₂0*s/L to 0 cm H₂0*s/L at approximately the time of 45 seconds. The exhalation resistance is then increased or set back to 20 cm H₂0*s/L thereafter.

FIG. 5 shows mouth pressure (e.g., measured in units of cm H₂0) on the left hand side Y-axis of graph 506 and time (e.g., measured in units of seconds) on X-axis of graph 506. For example, the mouth pressure is measured at a patient interface (e.g., mask or mouthpiece 111) and is also referred to as Pₘₒᵤₜₕ. Referring to graph 506, for the time period between 20 seconds and 45 seconds, when the exhalation resistance is maintained at 20 cm H₂0*s/L, the mouth pressure, Pₘₒᵤₜₕ remained constant. When the exhalation resistance is reduced/dropped from 20 cm H₂0*s/L to 0 cm H₂0*s/L at approximately the time of 45 seconds, the mouth pressure, Pₘₒᵤₜₕ is lowered as can be clearly seen in graph 506. When exhalation resistance is increased or set back to 20 cm H₂0*s/L thereafter, the mouth pressure, Pₘₒᵤₜₕ is also increased to its previous value (i.e., the value of Pₘₒᵤₜₕ during the time period between 20 seconds and 45 seconds.

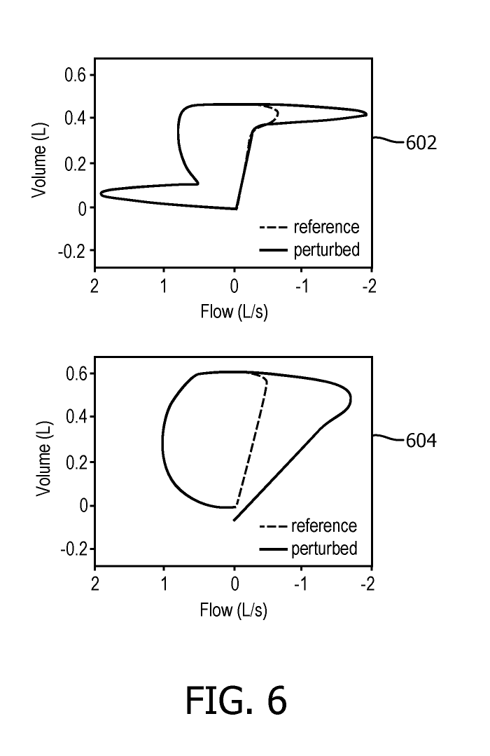

FIG. 6 shows two exemplary flow comparisons between perturbed and reference breaths (flow-volume curves/loops) obtained from system 100.

FIG. 6 shows flow information (e.g., measured in units of liters/second) on the X-axis of flow-volume curves 602 and 604. FIG. 6 also shows volume information (e.g., measured in units of liters) on the left Y-axis of flow-volume curves 602 and 604.

In the left plot of FIG. 6 or flow-volume curve 602, an increase in expiratory drive does not cause an increase in flow. That is, the dotted line (reference expiratory flow-volume) curve and line (perturbed expiratory flow-volume) curve are very close to each other (except for the very beginning of the expiratory phase). Thus, the breath in the left plot of FIG. 6 is flow-limited. The flow-volume curve 602 shows a flow-volume curve with the EFL.

In the right plot of FIG. 6 or flow-volume curve 604, an increase in expiratory drive causes a significantly higher exhalation flow. That is, the dotted line (reference expiratory flow-volume) curve and line (perturbed expiratory flow-volume) curve are not close to each other. Thus, the breath in the right plot of FIG. 6 or flow-volume curve 604 is not flow-limited. The flow-volume curve 604 shows a flow-volume curve without the EFL.

FIG. 7 shows an example of EFL detection by reduction of exhalation resistance in system 100 and corresponding treatment to abolish EFL. In one embodiment, following detection of EFL, the expiratory pressure level is increased by increasing the exhalation resistance and the procedure (perturbation, classification algorithm and resistance update) is repeated.

FIG. 7 shows exhalation resistance (e.g., measured in units of cm H₂0*s/L) on the left hand side Y-axis of graph 702 and time (e.g., measured in units of seconds) on X-axis of graph 702. For example, the exhalation resistance is the flow resistance applied by flow resistor 110 in exhalation path 108. Referring to graph 702, for the time period between 0 seconds and 25 seconds, the exhalation resistance is maintained at 15 cm H₂0*s/L. In one embodiment, the airflow waveforms/flow-volume curves from the reference breaths (e.g., one or multiple breaths preceding the perturbed breath) are sent to the classification algorithm (e.g., Expiratory Flow Limitation detection subsystem 118) from reference expiratory flow-volume curve determination subsystem 112, for example, during the time period between 0 seconds and 25 seconds as shown in FIG. 7.

The exhalation resistance is then reduced or dropped from 15 cm H₂0*s/L to 0 cm H₂0*s/L at approximately the time of 25 seconds. In one embodiment, the airflow waveforms/flow-volume curves from the perturbed breath are sent to the classification algorithm (e.g., Expiratory Flow Limitation detection subsystem 118) from perturbed expiratory flow-volume curve determination subsystem 116, for example, after the time period of 25 seconds as shown in FIG. 7. In one embodiment, Expiratory Flow Limitation detection subsystem 118 is configured to detect the Expiratory Flow Limitation (EFL) of the patient by comparing the determined perturbed expiratory flow-volume curve with the determined reference expiratory flow-volume curve.

In one embodiment, if the Expiratory Flow Limitation (EFL) of the patient is detected, the exhalation resistance is increased to, for example, 17 cm H₂0*s/L, for example, during the time period between 32 seconds and 47 seconds as shown in FIG. 7. The procedures repeat thereafter. That is, for the time period between 32 seconds and 47 seconds, the exhalation resistance is maintained at 17 cm H₂0*s/L. The exhalation resistance is then reduced or dropped from 17 cm H₂0*s/L to 0 cm H₂0*s/L at approximately after the time of 47 seconds. In one embodiment, the airflow waveforms/flow-volume curves from the reference breaths (e.g., one or multiple breaths preceding the perturbed breath) are sent to the classification algorithm (e.g., expiratory Flow Limitation Detection subsystem 118) from reference expiratory flow-volume curve determination subsystem 112, for example, during the time period between 32 seconds and 47 seconds as shown in FIG. 7. In one embodiment, the airflow waveforms/flow-volume curves from the perturbed breath are sent to the classification algorithm (e.g., expiratory Flow Limitation Detection subsystem 118) from perturbed expiratory flow-volume curve determination subsystem 116, for example, after the time period of 47 seconds as shown in FIG. 7. In one embodiment, Expiratory Flow Limitation detection subsystem 118 is configured to detect the Expiratory Flow Limitation (EFL) of the patient by comparing the determined perturbed expiratory flow-volume curve with the determined reference expiratory flow-volume curve.

In one embodiment, if the Expiratory Flow Limitation (EFL) of the patient is not detected, the exhalation resistance is decreased/reduced. In one embodiment, if the Expiratory Flow Limitation (EFL) of the patient is not detected, the exhalation resistance is not increased.

FIG. 7 also shows volume (e.g., measured in units of liters) on the left hand side Y-axis of graph 704 and time (e.g., measured in units of seconds) on X-axis of graph 704. For example, the volume is the flow-volume information obtained from sensor 106.

FIG. 8 shows a more detailed flow chart for the embodiment in FIG. 7. FIG. 8 shows an example of EFL detection by exhalation resistance reduction and corresponding abolishment flow chart. FIG. 8 is a flow chart for detecting EFL of the patient. Referring to FIG. 8, method 800 for detecting EFL of the patient is provided. Method 800 is implemented by computer system 102 that comprises one or more physical processors executing computer program instructions which, when executed, perform method 800. Method 800 comprises: obtaining, from one or more sensors (106), flow-volume information of exhaled air through exhalation passage 108; determining, by computer system 102, a reference expiratory flow-volume curve using flow-volume information of the exhaled air through exhalation passage 108 when a reference exhalation resistance is provided by flow resistor 110 in exhalation passage 108; adjusting flow resistor 110 to lower the exhalation resistance below the reference exhalation resistance; determining, by computer system 102, a perturbed expiratory flow-volume curve using flow-volume information of the exhaled air through exhalation passage 108 when the lowered exhalation resistance is provided by flow resistor 110 in exhalation passage 108; and detecting, by computer system 102, the Expiratory Flow Limitation (EFL) of the patient based on (i) the determined perturbed expiratory flow-volume curve (ii) the determined reference expiratory flow-volume curve.

In one embodiment, referring to FIG. 8, system 100 is started with a set/reference exhalation resistance at procedure 801. In one embodiment, at procedure 802, system 100 is continued to operate or run at the set/reference exhalation resistance, for example, for n breaths. In one embodiment, at procedure 803, system 100 is configured to change the exhalation resistance (i.e., from the set/reference exhalation resistance to a different exhalation resistance) in exhalation passage 108 (e.g., by adjusting flow resistor 110) during the exhalation phase of (n+1) breath.

In one embodiment, at procedure 804, system 100 is configured to change the exhalation resistance in exhalation passage 108 (e.g., by adjusting flow resistor 110) on exhalation phase of (n+2) breath. In one embodiment, at procedure 804, system 100 is configured to change the exhalation resistance in exhalation passage 108 to the set/reference exhalation resistance.

In one embodiment, at procedure 805, system 100 is configured to determine reference expiratory flow-volume curve using the flow-volume information of the exhaled air through exhalation passage 108 for the n breaths. In one embodiment, at procedure 805, system 100 is also configured to determine perturbed expiratory flow-volume curve using the flow-volume information of the exhaled air through exhalation passage 108 for the n+1 breath. In one embodiment, at procedure 805, system 100 is configured to detect the Expiratory Flow Limitation (EFL) of the patient based on (i) the determined perturbed expiratory flow-volume curve (ii) the determined reference expiratory flow-volume curve.

In one embodiment, at procedure 806, if the EFL is detected, then, at procedure 807, system 100 is configured to increase the set exhalation resistance in exhalation passage 108 (e.g., by adjusting flow resistor 110). In one embodiment, method 800 loops/goes back to procedure 802 after procedure 806 and method 800 repeats therefrom.

In one embodiment, at procedure 806, if the EFL is not detected, then, at procedure 808, system 100 is configured to operate at the set/reference exhalation resistance in exhalation passage 108 for n breaths.

In one embodiment, at procedure 809, system 100 is configured to determine whether the EFL is detected for x consecutive times. In one embodiment, if the EFL is not detected for x consecutive times, then method 800 loops/goes back to procedure 802 after procedure 809 and method 800 repeats therefrom.

In one embodiment, if the EFL is detected for x consecutive times, then, at procedure 810, system 100 is configured to reduce the set/reference exhalation resistance in exhalation passage 108 (e.g., by adjusting flow resistor 110). In one embodiment, method 800 loops/goes back to procedure 802 after procedure 810 and method 800 repeats therefrom.

In one embodiment, the patient goes to a doctor. In one embodiment, the patient's doctor asks questions about, for example, 1) history of patient's smoking; 2) exposure to secondhand smoking, air pollution, chemical or dust; 3) symptoms such as shortness of breath, chronic cough and mucus, etc. In one embodiment, the patient's doctor performs spirometry test to determine first Forced expiratory volume (FEV1) and Forced vital capacity (FVC). In one embodiment, the patient's doctor, using that information, determines whether the patient has a COPD and determines the patient's COPD stage classification.

In one embodiment, the patient's doctor then uses system 100 of the present patent application to detect EFL in that COPD patient. In one embodiment, the patient's doctor uses system 100 to directly assess whether the patient is affected by EFL. In one embodiment, the patient is asked by the doctor to normally breathe through system 100 in the supine position. In one embodiment, system 100 includes a resistance on exhalation path 108 that creates positive exhalation pressure (PEP). That is, in one embodiment, system 100 causes PEP by means of its exhalation resistance. In one embodiment, once the patient is comfortably breathing through system 100, the patient's doctor presses a (manual or electronic) button that removes the exhalation resistance on exhalation path 108. That is, after a few breaths, one breath is taken without the exhalation resistance. That is, the patient takes a few breaths at higher PEP followed by a few breaths at lower PEP. In one embodiment, the PEP may be changed manually. In one embodiment, flow is measured and flow-volume loops are used to compare consecutive breaths at different PEP.

In one embodiment, the patient's doctor is presented, by system 100, flow-volume plots or curves, for example, such as shown in FIG. 6. That is, referring to FIG. 6, exhalation flow-volume loops for breaths with exhalation resistance (reference) and without exhalation resistance (perturbed) are compared. The latter corresponds to breaths with increased expiratory pressure drive. If they do not lead to increased flow, the patient is flow limited.

In one embodiment, the patient's doctor then prescribes vector/therapy if the patient is flow-limited. That is, if the patient's flow-volume plots are similar to that shown in the left hand side plots in FIG. 6, the patient's doctor determines that the patient is affected by EFL and then prescribes vector/therapy.

In one embodiment, system 100 includes a (transmitting) unit to transmit the measured flow information (or flow-volume curve(s)) to an external processor and/or display (e.g., smartphone, tablet or dedicated processor/display) for the clinician to analyze the flow-volume curve(s) and make the diagnosis.

In one embodiment, system 100 provides a portable, inexpensive device or system for the diagnosis of EFL in non-ventilated patients. In one embodiment, system 100 requires no collaboration from the patient.

In one embodiment, system 100 may be used for EFL screening in a doctor's office. In one embodiment, system 100 may be used for EFL monitoring at a patient's home. In one embodiment, system 100 may be used for more comfortably assessing pharmaceutical treatments.

In one embodiment, system 100 may be used for on-line EFL detection. In one embodiment, one or more processors of computer system 102 (e.g., running an algorithm) are configured to automatically perform changes in exhalation pressure/resistance on select breaths. In one embodiment, changes in exhalation pressure on select breaths are manually triggered by the patient.

In one embodiment, one or more processors of computer system 102 are configured to compute flow-volume curves. In one embodiment, one or more processors of computer system 102 are configured (e.g., by running an algorithm) for the automated classification (i.e., flow-limited vs. non-flow-limited) of the breaths corresponding to such curves. In one embodiment, the output of the algorithm (i.e., EFL or no EFL) is signaled to the patient by visual or audio signals. In one embodiment, system 100 includes a (transmitting) unit to transmit the measured flow information (or flow-volume curve(s)) for remote monitoring and/or for the patient to have access to the actual flow-volume curves processed by the classification algorithm.

In one embodiment, the alteration of the expiratory resistance comprises, after a plurality of breaths at reference resistance (or reference positive expiratory pressure), changing the resistance, typically to a lower level, and maintaining the new resistance (or positive expiratory pressure) for one or more breaths. In one embodiment, the expiratory resistance is continuously adjusted during the expiratory phase using feedback control and/or adaptive feed-forward control or compensation.

In one embodiment, a subsystem of system 100 may be configured to determine the reference exhalation resistance using previously obtained pressure information, previously obtained flow information, previously obtained exhalation resistance information, previously obtained flow-volume information and/or previously obtained EFL information from a plurality of patients. In one embodiment, this subsystem is also configured to continuously obtain subsequent pressure information, subsequent flow information, subsequent flow-volume information, subsequent exhalation resistance information, and/or subsequent EFL information of the plurality of patients. That is, the subsystem may continuously obtain subsequent information associated with the multiple patients. As an example, the subsequent information may comprise additional information corresponding to a subsequent time (after a time corresponding to information that was used to determine the EFL information). As an example, the subsequent information may be obtained from one or more monitoring devices and associated one or more sensors.

The subsequent information may be utilized to further update or modify the reference/set exhalation resistance (e.g., new information may be used to dynamically update or modify the reference/set exhalation resistance), etc. In some embodiments, this subsystem is configured to then continuously modify or update the reference/set exhalation resistance based on the subsequent pressure information, the subsequent flow information, subsequent flow-volume information, the subsequent exhalation resistance information, the subsequent EFL information or other subsequent information. For example, the “subsequent” information may be used in addition to the flow-volume loops (e.g., as described herein) to determine whether a patient is flow-limited.

In one embodiment, the present patent application provides an inexpensive (low cost) and portable system/device for the detection and treatment of EFL. In one embodiment, system 100 is portable and inexpensive (as opposed to the Forced Oscillation Technique (FOT) and Negative Expiratory Pressure (NEP) devices). In one embodiment, system 100 is a hand-held device.

Thanks to its simplicity, system 100 of the present patent application can be used for a wide range of applications, from screening in a doctor's office, to on-line detection of EFL on patients who are regularly using the device/system to relieve the EFL symptoms and even on-line detection combined with automatic adjustment of the device/system itself to abolish EFL. In one embodiment, system 100 of the present patent application can be used for periodic monitoring/diagnosis at home as well as continuous real-time detection and abolishment of EFL.

In one embodiment, system 100 is attractive because system 100 is configured to detect EFL in a direct way. That is, system 100 is configured to measure EFL according to the definition of EFL instead of relying on measurements more or less correlated to EFL (e.g., like ΔXrs and FEV1/FVC measurements, obtained via a Forced Oscillation Technique (FOT) and spirometry, respectively).

In one embodiment, system 100 is configured to provide direct assessment of EFL. In one embodiment, the detection by system 100 is based on the same principle of the current practice of manual abdomen compression, but it is automated to overcome variability and subjectivity of manual procedures.

In one embodiment, system 100 does not require patient's collaboration (as opposed to spirometry). In one embodiment, system 100 can be designed with different levels of automation to meet different needs, from screening to prolong use with detection and abolishment of EFL.

In one embodiment, the various computers and subsystems illustrated in FIG. 2 may comprise one or more computing devices that are programmed to perform the functions described herein. The computing devices may include one or more electronic storages (e.g., database 132, or other electronic storages), one or more physical processors programmed with one or more computer program instructions, and/or other components. The computing devices may include communication lines or ports to enable the exchange of information with a network (e.g., network 150) or other computing platforms via wired or wireless techniques (e.g., Ethernet, fiber optics, coaxial cable, WiFi, Bluetooth, near field communication, or other communication technologies). The computing devices may include a plurality of hardware, software, and/or firmware components operating together to provide the functionality attributed herein to the servers. For example, the computing devices may be implemented by a cloud of computing platforms operating together as the computing devices.

The electronic storages may comprise non-transitory storage media that electronically stores information. The electronic storage media of the electronic storages may include one or both of system storage that is provided integrally (e.g., substantially non-removable) with the servers or removable storage that is removably connectable to the servers via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storages may store software algorithms, information determined by the processors, information received from the servers, information received from client computing platforms, or other information that enables the servers to function as described herein.

The processors may be programmed to provide information processing capabilities in the servers. As such, the processors may include one or more of a digital processor, an analog processor, or a digital circuit designed to process information, an analog circuit designed to process information, a state machine, and/or other mechanisms for electronically processing information. In one embodiment, the processors may include a plurality of processing units. These processing units may be physically located within the same device, or the processors may represent processing functionality of a plurality of devices operating in coordination. The processors may be programmed to execute computer program instructions to perform functions described herein of subsystems 112-120 or other subsystems. The processors may be programmed to execute computer program instructions by software; hardware; firmware; some combination of software, hardware, or firmware; and/or other mechanisms for configuring processing capabilities on the processors.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the present patent application has been described in detail for the purpose of illustration based on what is currently considered to be the most practical and preferred embodiments, it is to be understood that such detail is solely for that purpose and that the present patent application is not limited to the disclosed embodiments, but, on the contrary, is intended to cover modifications and equivalent arrangements that are within the spirit and scope of the appended claims. For example, it is to be understood that the present patent application contemplates that, to the extent possible, one or more features of any embodiment can be combined with one or more features of any other embodiment.

What is claimed is:

1. A system (100) for detecting an Expiratory Flow Limitation (EFL) of a patient, the system comprising:
an inhalation passage (104) that brings inhaled air to the patient;
an exhalation passage (108) that takes exhaled air away from the patient;
a sensor (106) for measuring flow-volume information of the exhaled air through the exhalation passage;
a flow resistor (110) positioned in the exhalation passage and being adjustable to provide an exhalation resistance in the exhalation passage; and
a computer system (102) that comprises one or more physical processors operatively connected with the sensor and the flow resistor, the one or more physical processors are programmed with computer program instructions which, when executed cause the computer system to:
determine a reference expiratory flow-volume curve using flow-volume information of the exhaled air through the exhalation passage when a reference exhalation resistance is provided by the flow resistor in the exhalation passage;
adjust the flow resistor to lower the exhalation resistance below the reference exhalation resistance;
determine a perturbed expiratory flow-volume curve using flow-volume information of the exhaled air through the exhalation passage when the lowered exhalation resistance is provided by the flow resistor in the exhalation passage; and
detect the Expiratory Flow Limitation (EFL) of the patient based on (i) the determined perturbed expiratory flow-volume curve and (ii) the determined reference expiratory flow-volume curve.

2. The system of claim 1, wherein the computer system detects the Expiratory Flow Limitation (EFL) of the patient by comparing the determined perturbed expiratory flow-volume curve with the determined reference expiratory flow-volume curve.

3. The system of claim 2, wherein the reference expiratory flow-volume curve is determined using the flow-volume information of the exhaled air through the exhalation passage for one or more reference breaths in which the reference exhalation resistance is provided by the flow resistor in the exhalation passage, wherein the perturbed expiratory flow-volume curve is determined using the flow-volume information of the exhaled air through the exhalation passage of a perturbed breath in which the lowered exhalation resistance is provided by the flow resistor in the exhalation passage, and wherein the one or more reference breaths are preceding the perturbed breath.

4. The system of claim 1, wherein the computer system automatically increases or decreases an exhalation airway pressure upon detection of the EFL in order to abolish the EFL.

5. The system of claim 3, wherein the comparing includes comparing the expiratory flow-volume curve for the one or more reference breaths with an expiratory flow-volume curve for one or more perturbed breaths.

6. A method (800) for detecting an Expiratory Flow Limitation (EFL) of a patient, the method being implemented by a computer system (102) that comprises one or more physical processors executing computer program instructions which, when executed, perform the method, the method comprising:
obtaining, from one or more sensors (106), flow-volume information of exhaled air through an exhalation passage (108);
determining, by the computer system, a reference expiratory flow-volume curve using flow-volume information of the exhaled air through the exhalation passage when a reference exhalation resistance is provided by a flow resistor (110) in the exhalation passage;
adjusting the flow resistor to lower the exhalation resistance below the reference exhalation resistance;
determining, by the computer system, a perturbed expiratory flow-volume curve using flow-volume information of the exhaled air through the exhalation passage when the lowered exhalation resistance is provided by the flow resistor in the exhalation passage; and
detecting, by the computer system, the Expiratory Flow Limitation (EFL) of the patient based on (i) the determined perturbed expiratory flow-volume curve (ii) the determined reference expiratory flow-volume curve.

7. The method of claim 6, wherein the detecting the Expiratory Flow Limitation (EFL) of the patient includes comparing the determined perturbed expiratory flow-volume curve with the determined reference expiratory flow-volume curve.

8. The method of claim 7, wherein the reference expiratory flow-volume curve is determined using the flow-volume information of the exhaled air through the exhalation passage for one or more reference breaths in which the reference exhalation resistance is provided by the flow resistor in the exhalation passage, wherein the perturbed expiratory flow-volume curve is determined using the flow-volume information of the exhaled air through the exhalation passage of a perturbed breath in which the lowered exhalation resistance is provided by the flow resistor in the exhalation passage, and wherein the one or more reference breaths are preceding the perturbed breath.

9. The method of claim 6, wherein the computer system automatically increases or decreases an exhalation airway pressure upon detection of the EFL in order to abolish the EFL.

10. The method of claim 7, wherein the comparing includes comparing the expiratory flow-volume curve for the one or more reference breaths with an expiratory flow-volume curve for one or more perturbed breaths.

11. A system (100) for detecting an Expiratory Flow Limitation (EFL) of a patient, the system comprising:
a means (102) for executing machine-readable instructions with at least one physical processor, wherein the machine-readable instructions comprising:
obtaining, from one or more sensors (106), flow-volume information of exhaled air through an exhalation passage (108);
determining a reference expiratory flow-volume curve using flow-volume information of the exhaled air through the exhalation passage when a reference exhalation resistance is provided by a flow resistor (110) in the exhalation passage;
adjusting the flow resistor to lower the exhalation resistance below the reference exhalation resistance;
determining a perturbed expiratory flow-volume curve using flow-volume information of the exhaled air through the exhalation passage when the lowered exhalation resistance is provided by the flow resistor in the exhalation passage; and
detecting the Expiratory Flow Limitation (EFL) of the patient based on (i) the determined perturbed expiratory flow-volume curve (ii) the determined reference expiratory flow-volume curve.

12. The system of claim 11, wherein the detecting the Expiratory Flow Limitation (EFL) of the patient includes comparing the determined perturbed expiratory flow-volume curve with the determined reference expiratory flow-volume curve.

13. The system of claim 12, wherein the reference expiratory flow-volume curve is determined using the flow-volume information of the exhaled air through the exhalation passage for one or more reference breaths in which the reference exhalation resistance is provided by the flow resistor in the exhalation passage, wherein the perturbed expiratory flow-volume curve is determined using the flow-volume information of the exhaled air through the exhalation passage of a perturbed breath in which the lowered exhalation resistance is provided by the flow resistor in the exhalation passage, and wherein the one or more reference breaths are preceding the perturbed breath.

14. The system of claim 11, the machine-readable instructions further comprising automatically increasing or decreasing an exhalation airway pressure upon detection of the EFL in order to abolish the EFL.

15. The system of claim 13, wherein the comparing includes comparing the expiratory flow-volume curve for the one or more reference breaths with an expiratory flow-volume curve for one or more perturbed breaths.
